(19) United States
(12) Patent Application Publication
(10) Pub. No.: US 2024/0301394 A1
(43) Pub. Date: Sep. 12, 2024

(54) DEVICES AND METHODS FOR ELECTROPHORETIC EXTRACTION OF NUCLEIC ACIDS FROM BIOLOGICAL SAMPLES

(71) Applicant: Roche Sequencing Solutions, Inc., Pleasanton, CA (US)

(72) Inventors: Yann ASTIER, Pleasanton, CA (US); Pantea GHEIBI, Pleasanton, CA (US); Jaeyoung YANG, Pleasanton, CA (US); Vladimira DATINSKA, Pleasanton, CA (US); Keynttisha JEFFERSON, Pleasanton, CA (US); Kevin VITTAYARUKSKUL, Pleasanton, CA (US)

(21) Appl. No.: 18/279,319

(22) PCT Filed: Mar. 3, 2022
(86) PCT No.: PCT/EP2022/055421

§ 371 Date: Aug. 29, 2023

Related U.S. Application Data
(60) Provisional application No. 63/155,940, filed on Mar. 3, 2021.

(51) Int. Cl. C12N 15/10

(52) U.S. Cl. CPC C12N 15/101; C12N 15/1017

(57) ABSTRACT

The invention relates to a device methods and an assembly for isolating biological polymers from a sample, the device comprising a top reservoir, a bottom reservoir, a collection chamber located between the top and the bottom reservoirs and operably connected to the top and bottom reservoirs, a sieving matrix capable of passing the biological polymers to be extracted, a semipermeable membrane not capable of passing the biological polymers to be extracted, and at least one set of a working electrode and a counter electrode.

### FIELD OF THE INVENTION

The invention related to the field of isolation of nucleic acids from biological samples. More specifically, the invention relates to instruments, systems and methods of isolating nucleic acid using electrophoresis.

### BACKGROUND OF THE INVENTION

Nucleic acid-based diagnostics are becoming routine clinical practice. For example, genetic analysis of tumors or cell-free tumor DNA present in blood is used to guide treatment by pointing out targeted therapies specific for a patient's tumor mutation profile. The discovery of fetal DNA in maternal blood has enabled early prenatal testing for a range of genetic and chromosomal abnormalities. Genomic testing involves analysis of nucleic acids from clinical samples. Varieties of clinical sample types are submitted for analysis including body fluids, fresh tissue, frozen tissue and Formalin-Fixed Paraffin-Embedded (FFPE) tissue. The former is characterized by a large volume where the nucleic acid may be present in trace amounts. The latter is especially challenging for nucleic acid isolation, as the preservation process is known to damage and fragment the nucleic acids. The downstream analytical steps include e.g., next-generation sequencing and other complex methods relying on sufficient amount of quality nucleic acids to deliver the desired sensitivity and specificity of the clinical assays.

With all the technological advances in recent decades, there is an urgent need for a more robust and rapid nucleic acids extraction system, which is capable of processing wide range of biological samples. The described invention aims to fill this need by providing an adaptable extraction system, suited for extraction of total nucleic acids (DNA and RNA) from commonly used types of clinical samples.

### SUMMARY OF THE INVENTION

The instant invention comprises devices, assemblies and methods for electrophoretic isolation of biological polymers including nucleic acids from biological samples.

In some embodiments, the invention is a device for isolating biological polymers from a sample comprising: a top reservoir, a bottom reservoir, a collection chamber located between the top and the bottom reservoirs and operably connected to the top and bottom reservoirs, a sieving matrix capable of passing the biological polymers to be extracted, a semipermeable membrane not capable of passing the biological polymers to be extracted, and at least one set of a working electrode and a counter electrode. In some embodiments, the working electrode is located in the top reservoir and the counter electrode is located in the bottom reservoir. In some embodiments, the collection chamber is cylindrical or conical. In some embodiments, the sieving matrix is placed in the top chamber, e.g., at the interface of the top chamber and the collection chamber. In some embodiments, the semi-permeable is placed in the collection chamber, e.g., at the interface of the collection chamber and the bottom reservoir. In some embodiments, the molecular weight cut-off (MWCO) of the semi-permeable membrane is greater than the MWCO of the sieving matrix.

In some embodiments, the device further comprises electrolyte buffers in the top and bottom reservoirs. In some embodiments, the top and bottom reservoirs comprise the same electrolyte. In some embodiments, the top reservoir comprises a buffer with the leading electrolyte and the bottom reservoir comprises a buffer with the trailing electrolyte.

In some embodiments, the invention is a method of extracting biological polymers from a sample comprising: loading a leading electrolyte in to the bottom reservoir and collection chamber of the device described in the preceding section; contacting a biological sample with a trailing electrolyte; placing the sample into the top reservoir of the device described in the preceding section; applying a voltage to the electrodes; collecting the contents of the collection chamber comprising the extracted biological polymers. In some embodiments, the sample is placed onto sieving matrix within the top chamber.

In some embodiments, the biological polymers are nucleic acids. In some embodiments, the method further comprises amplifying and/or sequencing the isolated nucleic acids.

In some embodiments, the voltage is applied at constant power. In some embodiments, the sample is concentrated, undergoes an extraction process, or a deparaffinization process prior to being applied to the device.

In some embodiments, the invention is an assembly for isolating biological polymers from a sample comprising: a plurality of top reservoirs, a plurality of bottom reservoirs matching the number of the top reservoirs, a plurality of collection chambers located between the top and the bottom reservoirs and operably connected to the top and bottom reservoirs, in each top reservoir, a sieving matrix capable of passing the biological polymers to be extracted, in each collection chamber, a semipermeable membrane not capable of passing the biological polymers to be extracted, and a set of a working electrode and a counter electrode in each top reservoir. In some embodiments, the assembly further comprises an automated dispenser for dispensing one or more samples into one or more reservoirs of the plurality of top reservoirs. In some embodiments, the assembly further comprises a module for amplifying nucleic acids. In some embodiments, the assembly further comprises a module for sequencing nucleic acids. In some embodiments, the assembly further comprises a transfer module for transferring the sample.

### BRIEF DESCRIPTION OF THE DRAWINGS

FIG. 1 is an overview of the device for electrophoretic extraction of biological polymers.

FIG. 2 shows the shape and exemplary dimensions of the collection chamber.

FIG. 3 is a diagram of an assembly system for electrophoretic extraction of biological polymers.

FIG. 4 is a diagram of an automated device for electrophoretic extraction of biological polymers.

FIG. 5 illustrates the steps of assembling a prototype device for electrophoretic extraction of biological polymers.

FIG. 6 shows the results of isolating nucleic acids using the device.

### DETAILED DESCRIPTION OF THE INVENTION

### Introduction

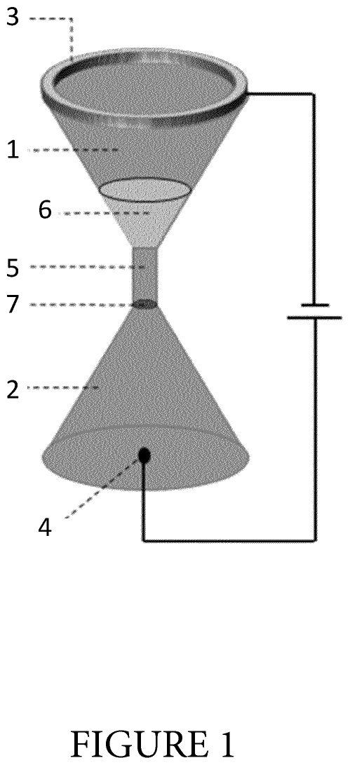

This invention describes devices integrating multiple components required for electrically driven extraction of biological polymers including nucleic acids (DNA and RNA) from a biological sample. Each device consists of components in an arrangement depicted in FIG. 1. Each component is explained below with respect to its function, geometry and/or material. The various aspects of the invention are described in further detail below.

### Sample

The present invention involves a method of extracting biological polymers such as nucleic acids from a sample. In some embodiments, the sample is derived from a subject or a patient. In some embodiments the sample may comprise a fragment of a solid tissue or a solid tumor derived from the subject or the patient, e.g., by biopsy. The sample may also comprise body fluids that may contain nucleic acids (e.g., urine, sputum, serum, blood or blood fractions, i.e., plasma, lymph, saliva, sputum, sweat, tear, cerebrospinal fluid, amniotic fluid, synovial fluid, pericardial fluid, peritoneal fluid, pleural fluid, cystic fluid, bile, gastric fluid, intestinal fluid, or fecal samples). In other embodiments, the sample is a cultured sample, e.g., a tissue culture containing cells and fluids from which nucleic acids may be isolated. In some embodiments, the nucleic acids of interest in the sample come from infectious agents such as viruses, bacteria, protozoa or fungi. In yet other embodiments, the sample is an environmental sample comprising solid or liquid material in which the biological polymers to be extracted are present.

In some embodiments, the sample is a solid sample selected from FFPET, fresh frozen tissue, needle biopsy. In some embodiments, the sample is a liquid sample selected from cultured cells, and bodily fluids such as plasma, blood, urine and saliva. In some embodiment, the sample applied to the electrophoretic device undergoes a pre-concentration step. In some embodiments, the sample, for example, a large-volume sample such as urine or blood plasma, is concentrated using e.g., centrifugation in a concentrator column such as Amicon column (Millipore, Sigma) to reduce the volume and concentrate the sample.

In some embodiments, prior to applying the sample to the electrophoretic device disclosed herein, the sample is pretreated to release of biopolymers from the associated molecules in the sample. In some embodiments, the pretreatments releases nucleic acids from proteins, lipids and phospholipids such as e.g., cellular membrane and nuclear membrane. Such treatments include but are not limited to deparaffinization of FFPET samples, protease digestion and cell lysis.

Methods of separating nucleic acids from other biological material are well known in the art. See J. Sambrook et al., “Molecular Cloning: A Laboratory Manual,” 1989, 2nd Ed., Cold Spring Harbor Laboratory Press: New York, N.Y.). A variety of kits are commercially available for extracting nucleic acids (DNA or RNA) from biological samples to form a solution of nucleic acids (e.g., KAPA Express Extract (Roche Sequencing Solutions, Pleasanton, Cal.) and other similar products from BD Biosciences Clontech (Palo Alto, Cal.), Epicentre Technologies (Madison, Wisc.); Gentra Systems, (Minneapolis, Minn.); and Qiagen (Valencia, Cal.), Ambion (Austin, Tex.); BioRad Laboratories (Hercules, Cal.); and more.

### Device

In some embodiments, the invention is a device integrating multiple components required for electrically driven extraction of biological polymers including nucleic acids (DNA and RNA) from a biological sample. Each device consists of components in an arrangement depicted in FIG. 1. Each component is explained below with respect to its function, geometry and material. In some embodiments, the device comprises a top reservoir 1 and a bottom reservoir 2. The top reservoir 1 contains a sample mixed with an electrolyte buffer. The bottom reservoir 2 contains an electrolyte buffer. The device further comprises set of working 3 and counter electrodes 4 between which the driving electric field forms. In some embodiments, the working electrode 3 is located in the top reservoir 1. In some embodiments, the working electrode 3 is located at or near the top rim of the top reservoir 1. In some embodiments, the counter electrode 4 is located in the bottom reservoir 2.

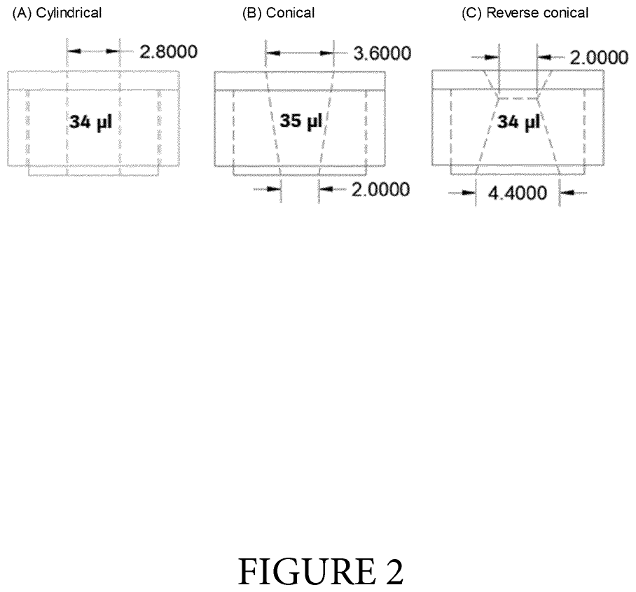

In some embodiments, the device further comprises a collection chamber 5 where extracted material is enriched for subsequent collection. In some embodiments, the collection chamber 5 is located between the top reservoir 1 and the bottom reservoir 2. In some embodiments, the collection chamber 5 has a body with openings on opposite side, wherein the topside opening is connected to the top reservoir 1 and the bottom-side opening is connected to the bottom reservoir 2. The collection chamber 5 can have different geometries as depicted in FIG. 2. In various embodiments, the collection chamber 5 is cylindrical (A) or conical (B-C). In some embodiments, the conical (B) collection chamber 5 has the wider base facing the top reservoir 1. In some embodiments, the conical (C) collection chamber 5 has the wider base facing the bottom reservoir 2 and is termed “reverse conical.” In some embodiments, the collection chamber 5 is configured to match any application-specific collection volume or the type of pipette tips used to withdraw the sample comprising isolated nucleic acids.

In some embodiments, the device comprises a sieving matrix or filter 6 for separation of biological polymers to be extracted from other components of the sample. In some embodiments, the sieving matrix or filer 6 is placed in the top reservoir 1. In some embodiments, the sieving matrix or filter 6 is placed at the interface of the top reservoir 1 and the collection chamber 5. The properties of the sieving matrix or filter 6 differ depending on the composition of the sample and the desired downstream procedure. In some embodiments, the dimensions of the sieving matrix or filter 6 are such that the sample nucleic acids travel through pores within the sieving matrix or filter 6. In some embodiments, the dimensions of the sieving matrix or filter 6 enable size-based exclusion of contaminating particles, e.g., cell debris, unlysed cells and other material larger than nucleic acids. In some embodiments, the dimensions of the sieving matrix or filter 6 enable minimizing pre-mix between two different buffers loaded into top and bottom reservoirs.

In some embodiments, the device comprises a semi-permeable membrane 7 for retaining biological polymers including nucleic acids. In some embodiments, the semi-permeable membrane 7 is placed in the collection chamber 5. In some embodiments, the semi-permeable membrane 7 is placed at the interface of the collection chamber 5 and the bottom reservoir 2. In some embodiments, the semi-permeable membrane 7 has a specific molecular weight cut-off (MWCO) for retaining the biological polymer to be extracted. In some embodiments, the MWCO of the semi-permeable membrane 7 is smaller than the MWCO of the sieving matrix or filter 6 in the collection chamber. In some embodiments, the semi-permeable membrane 7 comprise a dialysis membrane or porous foil and includes one or more of porous plastic materials, frits, gels, paper, nonwoven textiles and filtration paper.

### Buffer Systems

In some embodiments, the electrophoretic device utilizes a one-electrolyte system where both reservoirs are filled with a background electrolyte (e.g., zone electrophoresis). In other embodiments, the electrophoretic device is used for a discontinuous electrolyte system where top and bottom reservoirs are filled with two distinct electrolyte buffers. In some embodiments, the electrophoretic device is used with the two-buffer system comprising the leading and trailing electrolytes used in epitachophoresis as described e.g., in US20200282392 Devices for sample analysis using epitachophoresis. In some embodiments, the electrophoretic device is used with the two-buffer system comprising the leading and trailing electrolytes used in isotachophoresis as described e.g., in US20190071661 Systems devices and methods for isotachophoresis.

### Operation of the Device

In some embodiments, the electrophoretic device utilizes a flow of electricity to electrically transport negatively charged nucleic acid molecules towards the positively charged electrode immersed in the electrolyte-filled bottom reservoir 2. In some embodiments, the electrophoretic device is operated under a constant electrical parameter. The constant electrical parameter is one of constant power, constant current or constant voltage.

### Assembly

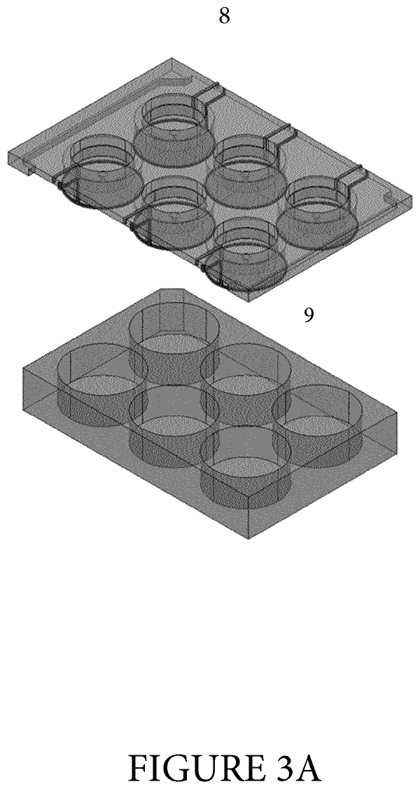

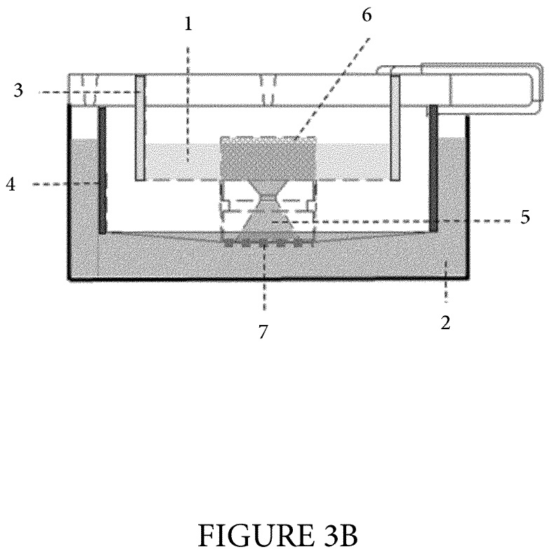

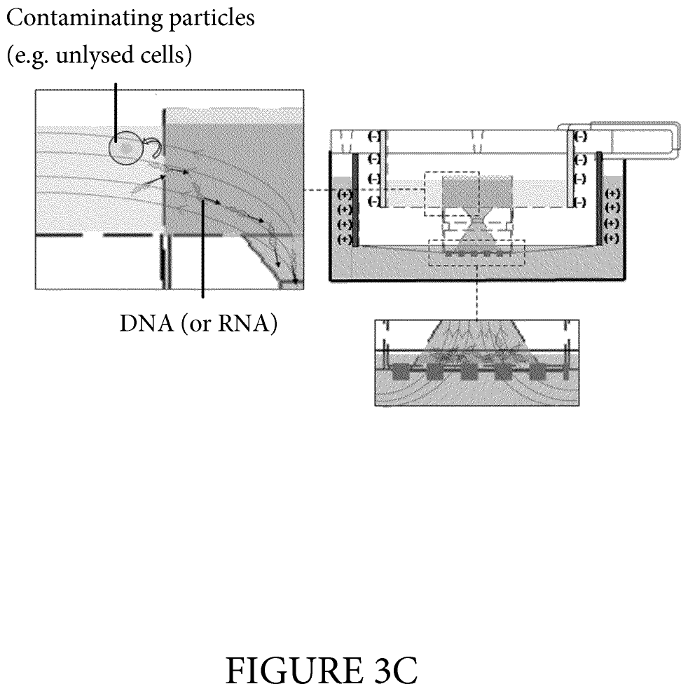

In some embodiments, the electrophoretic device is part of an assembly system for isolating nucleic acids described in FIGS. 3A and 3B. In some embodiments, the assembly device comprises top 8 and bottom plates 9. The top plate 8 includes electrodes 3, 4, filter 6, and membrane 7 within a reservoir and collection chamber 5. The top plate 8 is assembled with a bottom well plate 9 as seen in FIGS. 3A and 3B.

### Operation of the Assembly

In the example of FIGS. 3A and 3B, the bottom reservoir 2 along with the collection chamber 5 and the inner side of the filter paper 6 is filled with a buffer containing leading electrolyte ions. The outer region of the filter paper 6 is then filled with a liquid sample pre-mixed P36741 with trailing electrolyte.

In some embodiments, the sample is concentrated prior to applying to the assembly. In other embodiments, a large-volume sample containing trace biological polymers to be isolated (e.g., cell-free nucleic acids) is concentrated to increase the concentration of cell-free nucleic acids. In some embodiments, prior to applying to the assembly, the sample has undergone an initial pretreatment procedure to disrupt any cellular and subcellular structures comprising the biological polymers to be isolated. In some embodiments, a sample containing cells is treated with detergents and proteases to release nucleic acids. In some embodiments, the sample is an FFPET sample, which has undergone deparaffinization procedure prior to applying to the assembly.

Next, an electrical voltage is applied between the working 3 and counter electrodes 4. Driven by the voltage, the dispersed, charged biological polymers migrate to the opposite-charge electrode along the generated electric field. For example, negatively charged nucleic acids migrate towards the cathode. In some embodiments, large contaminating particles present in the sample such as unlysed cells or cell debris are prevented from diffusing through pores in the filter 6 with an appropriate pore size. At the same time, the biological polymers to be extracted pass through the filter 6 and reach a collection chamber 5. In some embodiments, smaller molecules constituting impurities pass through semipermeable membrane 7. At the same time, the biological polymers to be extracted have a molecular weight larger than MWCO of the semipermeable membrane 7 and are retained and enriched within the collection chamber 5 (FIG. 3C). The enriched material is then collected via manual or automated pipetting and transferred for subsequent sample processing and analysis.

### Automation

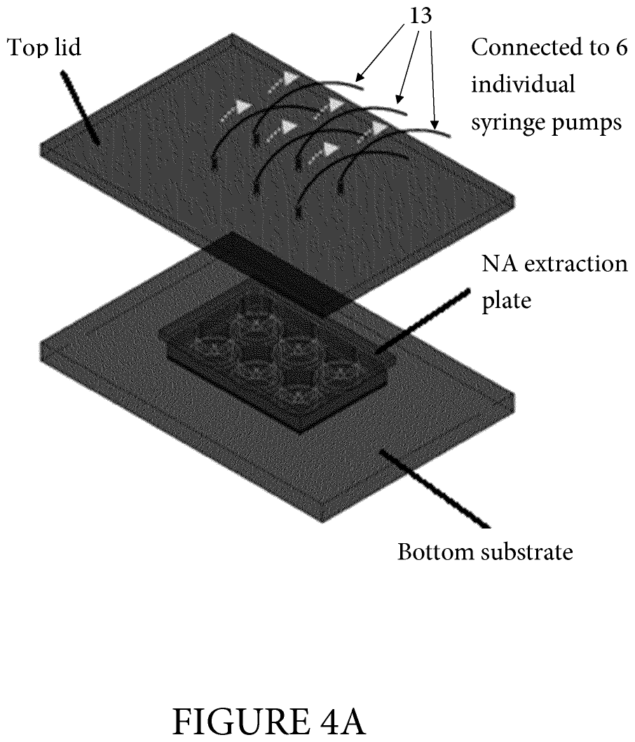

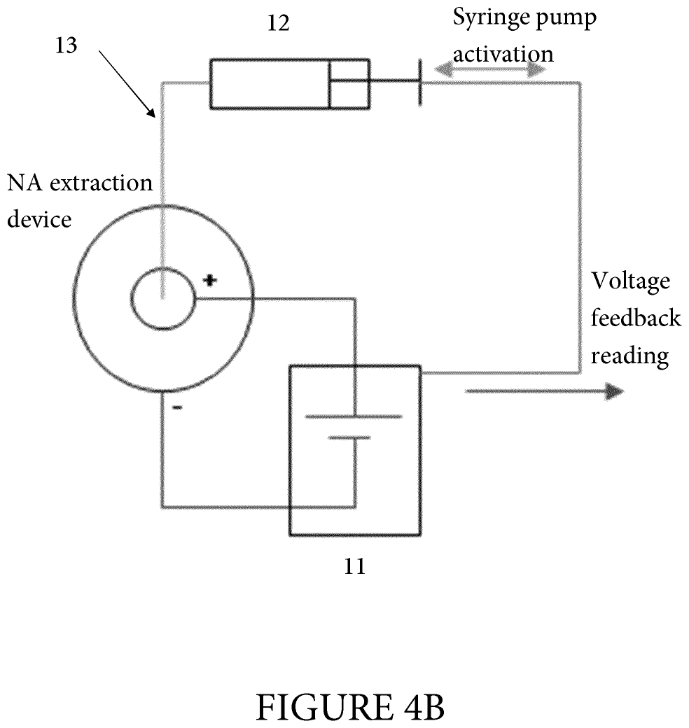

In some embodiments, isolation of biological polymers using the electrophoretic device is automated. The technology can be operated under an instrument to automate the workflow for improved throughput and reproducibility. A fully automated workflow can be implemented under a liquid handling robot for pipetting, and electrical and thermal control. In some embodiments, the automated device is assembled using standard culture plates, e.g., 6-well, 12-well 96-well or similar culture plates. For illustration, a simple fixture is shown in FIG. 4. As shown in FIG. 4, a 6-well extraction plate 8,9 loaded with samples and electrolyte buffers is first placed into the bottom substrate 10. The bottom substrate 10 houses a number (e.g., six in the example in FIG. 4) programmable power supplies 11 to provide the electrical power to individual wells. The power supply 11 also reads the feedback voltage from each well during the process. Once the voltage reaches a pre-defined cutoff value, the power is automatically reduced or shut down, which then activates the syringe pump 12 to aspirate the enriched nucleic acids from the collection chamber 5 over fluid lines 13.

### Assembling the Device

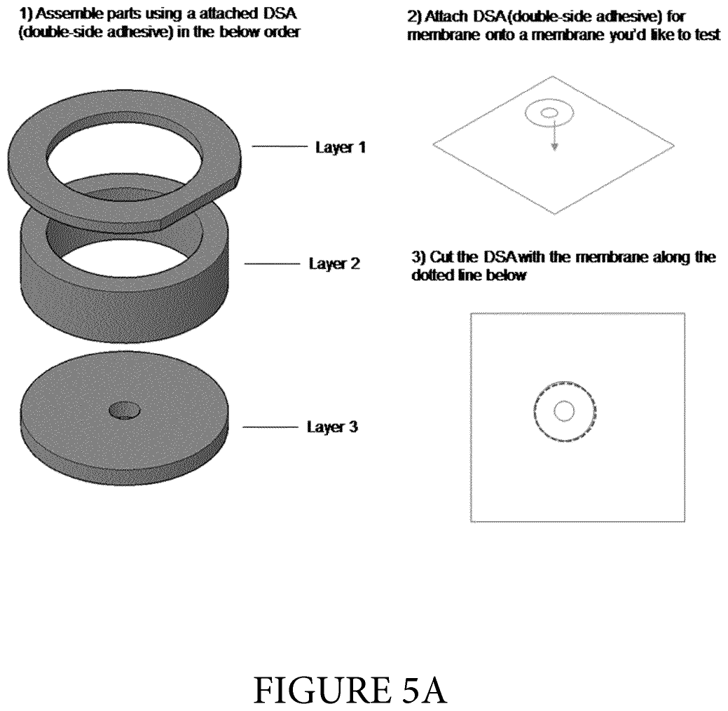

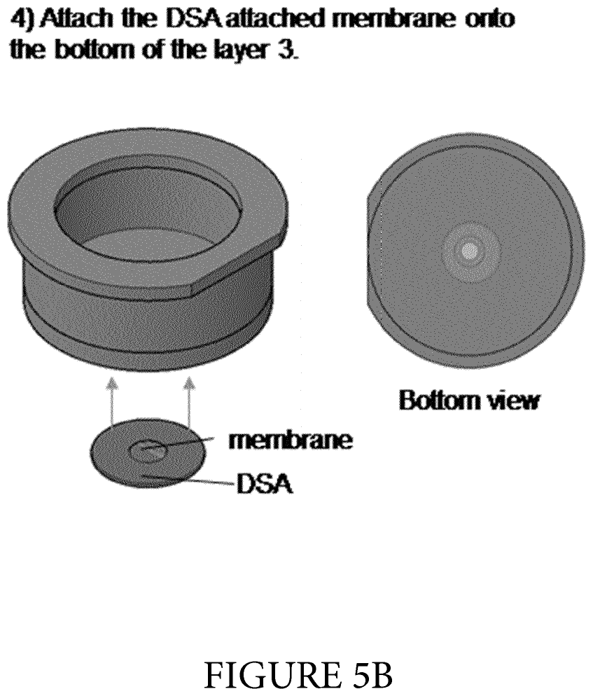

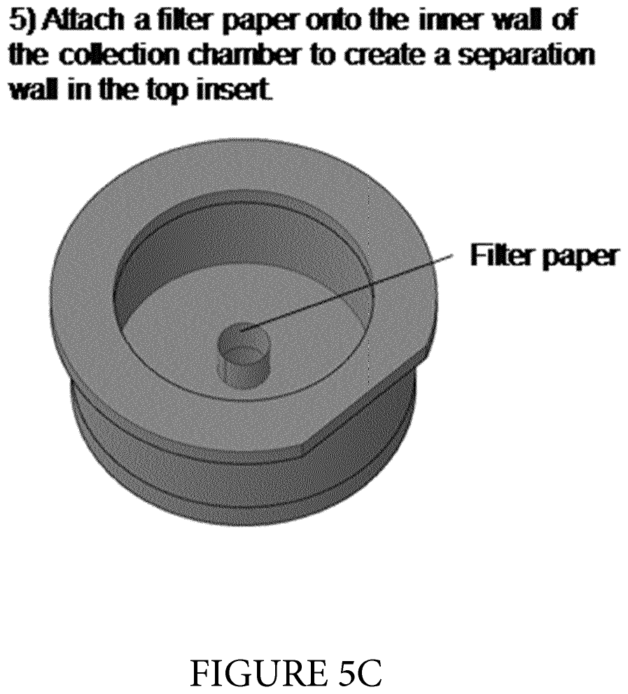

In some embodiments, the device is assembled from one or more layers of moldable material such as plastic, as shown in FIG. 5. In some embodiments, multiple moldable layers are laminated, as seen left in FIG. 5A, wherein layer 2 provides the sidewalls of the top reservoir 2 and layer 3 the bottom of the top reservoir 2 and the collection chamber 5 of a top insert. In some embodiments, the semi permeable membrane 7 is placed into the bottom of the device at the bottom opening of the collection chamber 5 wherein the semi permeable membrane 7 is shaped to accommodate the shape of each chamber (e.g., well), as can be seen in FIG. 5B (left: bottom view, right: perspective view with semi permeable membrane 7 exploded). A filter 6 is arranged surrounding the opening of the collection chamber 5, as shown in FIG. 5C. In some embodiments, the thickness and particle retention of the filter 6 is selected to accommodate the nature of the sample. In some embodiments, the particle retention is 25 μm. In some embodiments, the thickness (height) of the layers is 5 mm.

### Method of Extracting Biological Polymers

To operate the device, the leading electrolyte is placed into the bottom reservoir 2 and the collection chamber 5 of the top insert. In some embodiments, the leading electrolyte buffer has a higher ionic strength and a lower pH than the trailing electrolyte. In some embodiments, for isolating nucleic acids, the leading electrolyte comprises 100 mM HCl·His buffer, pH 6.25. Next, a sample solution comprising biological polymers (e.g., nucleic acids) is contacted to the hollow reservoir. In some embodiments, the sample is contacted to the filter 6 located in the top reservoir. In some embodiments, the sample is loaded into the outer side of the filter paper wall 6 within the top insert in the top reservoir.

In some embodiments, the sample solution further comprises one or more tracking dyes. In some embodiments, the tracking dye (e.g., brilliant blue) solution is prepared in trailing electrolyte. In some embodiments, the trailing electrolyte buffer has a lower ionic strength and a higher pH compared to the leading electrolyte buffer. In some embodiments, for isolating nucleic acids, the trailing electrolyte comprises 20 mM Taps·Tris, pH 8.30.

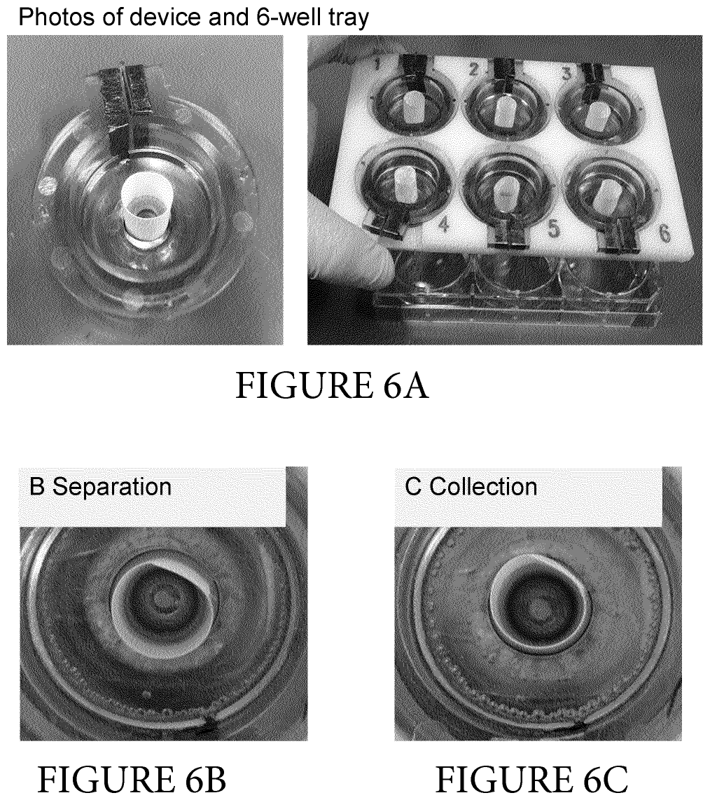

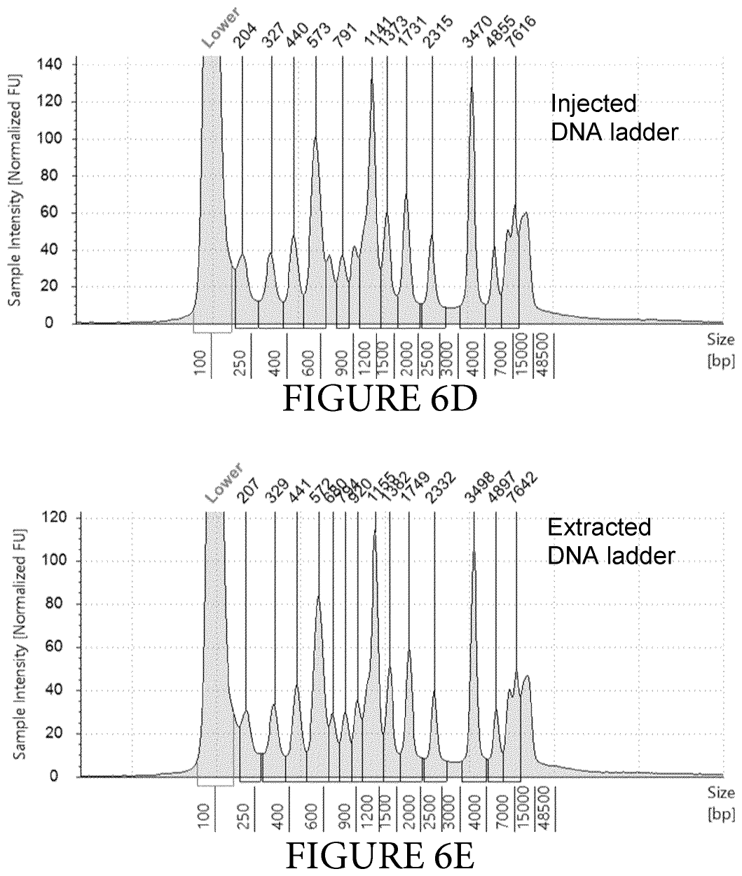

In some embodiments, to perform isolation of biological polymers, the device is run on constant power. In other embodiments, constant voltage or constant current may be used. The power drives the polymers and the tracking dye into the collection chamber 5 (FIG. 6A). The fractions collected from the collection chamber are retrieved for further analysis. In some embodiments, the collection is timed based on the migration of tracking dyes.

### Detection of Extracted Nucleic Acids

In some embodiments, the invention includes a step of detecting, qualifying or quantifying the extracted nucleic acids via amplification by polymerase chain reaction (PCR). The amplification utilizes an upstream primer and a downstream primer. In some embodiments, both primers are target specific primers, i.e., primers comprising a sequence complementary to a sequence known to be present in the extracted nucleic acids. In other embodiments, one or both primers are a mixture of random primers. In some embodiments, the PCR is real-time PCR also known as quantitative PCR. The qPCR utilizes a fluorescent probe wherein the level of fluorescence reflects the amount of the target nucleic acids in the reaction mixture. In some embodiments, qPCR is used to detect, qualify or quantify the amount of nucleic acids extracted with the method and apparatus disclosed herein.

In some embodiments, the invention includes a step of detecting the biological polymers extracted with the method and apparatus disclosed herein using fluorescence specific to the biological polymer. In some embodiments, the invention includes a step of analyzing the extract with a fluorimeter capable of reading the absorption of light at 260 nm and 280 nm and determining the 260/280 absorption ratio characteristic of proteins and nucleic acids.

### Sequencing

In some embodiments, the nucleic acids isolated and extracted with the method and apparatus disclosed herein are subjected to sequencing. In some embodiments, the isolated or extracted nucleic acids are amplified prior to sequencing. Currently used sequencing methods and platforms require forming a sequencing library. The library comprises a plurality of nucleic acids connected to platform-specific adaptors. Adaptors of various shapes and functions are known in the art (see e.g., WO2019/166565A1, U.S. Pat. Nos. 8,822,150 and 8,455,193). In some embodiments, the function of an adaptor is to introduce desired elements into a nucleic acid. The adaptor-borne elements include at least one of nucleic acid barcode, primer binding site or a ligation-enabling site. Commercially available kits for preparing nucleic acids, performing adaptor ligation to form a library and amplifying the library include AVENIO ctDNA Library Prep Kit or KAPA HyperPrep and HyperPlus kits (Roche Sequencing Solutions, Pleasanton, CA). In some embodiments, adaptors or amplification primers introduce barcode sequences. The use of molecular barcodes and sample barcodes in the sequencing process is described in U.S. Pat. Nos. 7,393,665, 8,168,385, 8,481,292, 8,685,678, 8,722,368 and WO2019/166565A1.

Non-limiting examples of sequence assays and platforms that are suitable for use with the methods disclosed herein include nanopore sequencing (U.S. Pat. Publ. Nos. 2013/0244340, 2013/0264207, 2014/0134616, 2015/0119259 and 2015/0337366), Sanger sequencing, capillary array sequencing, thermal cycle sequencing (Sears et al., Biotechniques, 13:626-633 (1992)), solid-phase sequencing (Zimmerman et al., Methods Mol. Cell Biol., 3:39-42 (1992)), sequencing with mass spectrometry such as matrix-assisted laser desorption/ionization time-of-flight mass spectrometry (MALDI-TOF/MS; Fu et al., Nature Biotech., 16:381-384 (1998)), sequencing by hybridization (Drmanac et al., Nature Biotech., 16:54-58 (1998), and NGS methods, including but not limited to sequencing by synthesis (e.g., HiSeq™, MiSeq™, or Genome Analyzer, each available from Illumina), sequencing by ligation (e.g., SOLID™, Life Technologies), ion semiconductor sequencing (e.g., Ion Torrent™, Life Technologies), and SMRT® sequencing (e.g., Pacific Biosciences).

Commercially available sequencing technologies include sequencing-by-hybridization platforms from Affymetrix Inc. (Sunnyvale, Calif.), sequencing-by-synthesis platforms from Illumina/Solexa (San Diego, Calif.) and Helicos Biosciences (Cambridge, Mass.), sequencing-by-ligation platform from Applied Biosystems (Foster City, Calif.). Other sequencing technologies include, but are not limited to, the Ion Torrent technology (ThermoFisher Scientific), and nanopore sequencing (Genia Technology from Roche Sequencing Solutions, Santa Clara, Cal.), and Oxford Nanopore Technologies (Oxford, UK).

### REFERENCES

U.S. Provisional Application Ser. No. 63/000,022, Devices and methods for sample analysis
US20200282392, Devices for sample analysis using epitachophoresis
WO2020/074742, Detection methods for Epitachophoresis workflow automation
WO2020/229437, Devices and methods for sample analysis
Foret, F. et al (2019) Macrofluidic Device for Preparative Concentration Based on Epitachophoresis Analytical Chemistry 91 (11), 7047-7053, DOI: 10.1021/acs.analchem.8b05860

### EXAMPLES

### Example 1. Isolating Nucleic Acids Using the Electrophoretic Device (Prophetic?)

In this example, the device shown in FIGS. 3A and 3B was used to isolate nucleic acids from an FFPET sample. The bottom reservoir 2 along with the collection chamber 5 and the inner side of the filter paper 6 are filled with a buffer containing leading electrolyte ions. The outer region of the filter paper 6 is then filled with a pre-treated FFPET sample mixed with a buffer containing trailing electrolyte ions. When an electrical voltage is applied between the working 3 and counter electrodes 4, dispersed, negatively charged nucleic acids in the sample migrate towards the positively-charged counter electrode 4 along the generated electric field. While large contaminating particles such as unlysed cells or cell debris are prevented from diffusing through pores in the filter paper 6, nucleic acid molecules pass through the structure and reach the collection chamber 5. Nucleic acids with a molecular weight larger than MWCO of the semipermeable membrane 7 are retained and enriched within the collection chamber 5 (FIG. 3C). The enriched nucleic acids are then collected via pipetting and transferred for subsequent sample processing and analysis.

### Example 2. Assembly of a Prototype Device for Electrophoretic Isolation of Nucleic Acids

In this example, to demonstrate the technological feasibility for NA extraction, we first prototyped a device in a single insert format which can be placed onto a standard 6-well culture plate. Multiple layers of plastic parts (layer 1, layer 2, layer 3) were cut using a laser cutting machine, and laminated using a double-sided adhesive, followed by attaching a semi-permeable membrane onto a bottom of the device as described in FIG. 5. A filter paper (VWR® Grade 415 Filter Paper, Qualitative, Crepe, Particle Retention 25 μm) was attached to the inner wall of the collection chamber to create a separation wall with a height of 5 mm in the top insert.

### Example 3. Testing the Prototype Device for Electrophoretic Isolation of Nucleic Acids

In this example, the prototype device of FIG. 6A was tested for epitachophoresis-based DNA extraction. A leading electrolyte solution (8.3 mL of 100 mM HCl·His buffer, pH 6.25) was first loaded into the bottom reservoir 2. The collection chamber 5 of the top insert was filled with 200 μL of the leading electrolyte solution. A sample solution containing DNA ladder (1 μg of 1 Kb plus, 100 bp-10 Kbp) and brilliant blue dye (as a tracking marker) prepared in a trailing buffer (1.0 mL of 20 mM Taps. Tris, pH 8.30) was then loaded into the outer side of the filter paper wall 6 within the top insert. A constant 0.5 W was applied to the device to drive the migration of DNAs and blue dye molecules into the collection chamber (FIG. 6A). The sample eluate collected after 8 min was then analyzed by Qubit to determine the extraction yield (i.e., recovery rate=75%, FIG. 6C). In addition, Tapestation results in FIG. 6D shows the comparable size distributions between sample input and output (i.e., eluate collected from the device), indicating that the device is capable of extracting DNAs regardless of their size.

While the invention has been described in detail with reference to specific examples, it will be apparent to one skilled in the art that various modifications can be made within the scope of this invention. Thus, the scope of the invention should not be limited by the examples described herein, but by the claims presented below.

1. A device for isolating biological polymers from a sample comprising:
a) a top reservoir,
b) a bottom reservoir,
c) a collection chamber located between the top and the bottom reservoirs and operably connected to the top and bottom reservoirs,
d) a sieving matrix capable of passing the biological polymers to be extracted,
e) a semi-permeable membrane not capable of passing the biological polymers to be extracted, and
f) at least one set of a working electrode and a counter electrode.

2. The device of claim 1, wherein the working electrode is located in the top reservoir.

3. The device of claim 1, wherein the counter electrode is located in the bottom reservoir.

4. The device of claim 1, wherein the collection chamber is cylindrical.

5. The device of claim 1, wherein the collection chamber is conical.

6. The device of claim 1, wherein the sieving matrix is placed in the top reservoir.

7. The device of claim 6, wherein the sieving matrix is placed at an interface of the top reservoir and the collection chamber.

8. The device of claim 1, wherein the semi-permeable membrane is placed in the collection chamber.

9. The device of claim 1, wherein the semi-permeable membrane is placed at an interface of the collection chamber and the bottom reservoir.

10. The device of claim 1, wherein the molecular weight cut-off (MWCO) of the semi-permeable membrane is greater than the MWCO of the sieving matrix.

11. The device of claim 1, further comprising electrolyte buffers in the top and bottom reservoirs.

12. The device of claim 11, wherein the top and bottom reservoirs comprise the same electrolyte.

13. The device of claim 11, wherein the top reservoir comprises a buffer with a leading electrolyte and the bottom reservoir comprises a buffer with a trailing electrolyte.

14. A method of extracting biological polymers from a sample comprising:
a. loading a leading electrolyte into a bottom reservoir and a collection chamber of a device, wherein the device further comprises:
a top reservoir,
a sieving matrix capable of passing the biological polymers to be extracted,
a semi-permeable membrane not capable of passing the biological polymers to be extracted, and
at least one set of a working electrode and a counter electrode, and wherein the collection chamber is located between the top and the bottom reservoirs and operably connected to the top and bottom reservoirs;
b. contacting a biological sample with a trailing electrolyte;
c. placing the sample into the top reservoir of the device;
d. applying a voltage to the at least one set of the working electrode and the counter electrodes;
e. collecting the contents of the collection chamber comprising extracted biological polymers.

15. The method of claim 14, wherein in step c., the sample is placed onto sieving matrix within the top chamber.

16. The method of claim 14, wherein the biological polymers are nucleic acids.

17-18. (canceled)

19. The method of claim 14, wherein in step d., the voltage is applied at constant power.

20. The method of claim 14, further comprising concentrating the sample prior to step b.

21-22. (canceled)

23. An assembly for isolating biological polymers from a sample comprising:
a) a plurality of top reservoirs,
b) a plurality of bottom reservoirs matching the number of the top reservoirs,
c) a plurality of collection chambers located between the top and the bottom reservoirs and operably connected to the top and bottom reservoirs,
d) in each top reservoir, a sieving matrix capable of passing the biological polymers to be extracted,
e) in each collection chamber, a semi-permeable membrane not capable of passing the biological polymers to be extracted, and
f) a set of a working electrode and a counter electrode in each top reservoir.

24. The assembly of claim 23, further comprising an automated dispenser for dispensing one or more samples into one or more reservoirs of the plurality of top reservoirs.

25-27. (canceled)
